(19) United States
(12) Patent Application Publication
(10) Pub. No.: US 2025/0352437 A1
(43) Pub. Date: Nov. 20, 2025

(54) Kit of Part for Producing Glass Ionomer Cement with High Compressive Strength

(71) Applicant: Solventum Intellectual Properties Company, Maplewood, MN (US)

(72) Inventors: Markus Mikulla, Andechs (DE); Peter Braun, Penzing (DE)

(21) Appl. No.: 18/874,400

(22) PCT Filed: May 31, 2023
(86) PCT No.: PCT/IB2023/055598

§ 371 Date: Dec. 12, 2024

(30) Foreign Application Priority Data
Jun. 28, 2022 (EP) 22181419.7

(51) Int. Cl. A61K 6/849; A61K 6/77

(52) U.S. Cl. CPC A61K 6/849; A61K 6/77

(57) ABSTRACT

The invention relates to a kit of parts for obtaining a glass ionomer composition, the kit of parts comprising Part P and Part L, Part P being a powder comprising acid-reactive glass, Part L being a liquid comprising water, the kit of parts containing in addition a polycarboxylic acid, the polycarboxylic acid being present in Part P or Part L or Part P and Part L, the polycarboxylic acid comprising a copolymer of acrylic acid and maleic acid, the acid-reactive glass being characterized by comprising P: 0-4 wt. %, F: 10-18 wt. %, O: 25-35 wt. %, Si: 10-16 wt. %, Al: 11-19 wt. %, Sr: 20-40 wt. %, La: 0-4 wt. % the combined amount of Al, Sr and F being >48 wt. %, wt. % with respect to the weight of the acid-reactive glass, and the ratio of Al and Si in the acid-reactive glass being greater than 1/1 with respect to weight. The invention also relates to the use of a particular acid reactive glass in combination with a particular polycarboxylic acid for improving the mechanical strength of a glass ionomer composition.

### FIELD OF THE INVENTION

The invention relates to a kit of parts for producing a glass ionomer composition having in particular beneficial mechanical properties, like compressive strength.

The glass ionomer composition can be used as filling material and for fixing dental restorations like dental crowns or bridges to tooth surfaces.

### BACKGROUND

Glass ionomer cements have been used for more than 30 years for dental restorative treatments.

Typically glass ionomer cements are reacted by mixing a powder part with a liquid part.

The powder component typically comprises as essential or important component an acid-reactive filler (e.g. a fluoroaluminosilicate glass).

The liquid component typically comprises as essential components water, polycarboxylic acid and a complexing or chelating agent (e.g. tartaric acid) for adjusting the setting properties.

Main advantages of glass ionomer cements are said to be self-adhesion to tooth structure, fluoride release and the ability to be placed in one part (bulk-fill).

One disadvantage reported by some practitioners is the brittle nature and relatively low physical-mechanical properties of the glass ionomer cement compared to the physical-mechanical properties reported for resin-based composite filling materials.

There have been various approaches to improve the mechanical properties of glass ionomer cements.

U.S. Pat. No. 4,376,835 (Schmitt et al.) describes a calcium aluminum fluorosilicate glass powder, wherein the calcium in the surface of the powder's particles is depleted. The glass powder may be prepared by surface treating calcium aluminum fluorosilicate powder particles with an acid which forms calcium salts, washing the calcium salts off the treated particles and drying the washed particles.

WO 2015/088956 A1 (3M IPC) relates to a kit of parts for preparing a glass ionomer cement, wherein the kit comprises a Part P and a Part L, Part P being a powder comprising an acid-reactive inorganic filler in a certain amount and having a mean particle size in the range of 3.5 to 10 μm, a non acid-reactive filler in a certain amount and having a mean particle size in the range of 1.0 to 3.5 μm, Part P not comprising polycarboxylic acid in an amount above 1 wt. %, Part L being a liquid and comprising a polycarboxylic acid in a certain amount, water and a complexing agent.

U.S. Pat. No. 10,080,708 B2 (3M) describes a kit of parts for preparing a glass ionomer cement, the kit comprising a Part A and a Part B, Part A being a powder and comprising an acid-reactive filler in an amount of above about 60 wt. % and having a mean particle size in the range of 3.5 to 10 μm, a non-acid reactive filler in an amount above about 1 wt. % and having a mean particle size in the range of 1 to 3.5 μm, Part B being a liquid and comprising a polyacid, water and a complexing agent. Useful acid-reactive glasses are said to have a Si/Al ratio (by wt. %) of below 1.5 or 1.4 or 1.3. Compressive strength values up to 271 MPa are reported.

U.S. Pat. No. 4,900,697 (GC) relates to a fluoroaluminosilicate glass powder for dental glass ionomer cements having a mean particle size of 0.02 to 10 μm and which consists essentially of 20 to 50 wt. % SiO₂, 20 to 40 wt. % of Al₂O₃, 15 to 40 wt. % of SrO, 1 to 20 wt. % F₂ and 0 to 15 wt. % P₂O₅, and is free from Li, Na, K, Rb, Cs, Be, Mg and Ba ions. For producing a glass ionomer cement composition, the glass powder is reacted with a polymer acid such as a polyacrylic acid, acrylic acid copolymer or polymaleic acid. Compressive strength values up to 237 MPa are reported.

WO 2021/049269 A1 (GC) describes a glass powder for a chemical polymerization initiator wherein the glass powder comprises aluminum, silicon and at least one of copper or vanadium for improving storage stability of a two-agent dental polymerizable composition. The ratio Al/Si of the glass used in the examples is >1.

### SUMMARY OF INVENTION

There is still a need for a glass ionomer composition having adequate mechanical properties, such as compressive strength and/or surface hardness, in particular after a short period of time.

Further, the glass ionomer composition should be easy to use, moisture tolerant, tooth coloured and biocompatible.

Ideally, the glass ionomer composition should be useful as an amalgam alternative and be essentially monomer free.

In one embodiment the present invention features a kit of parts as described in the present text and claims.

The kit of parts comprises Part P and Part L, Part P being a powder comprising acid-reactive glass, Part L being a liquid comprising water,
the kit of parts containing in addition a polycarboxylic acid, the polycarboxylic acid being present in Part P or Part L or Part P and Part L, the polycarboxylic acid comprising a copolymer of acrylic acid and maleic acid,
the acid-reactive glass being characterized by comprising
P: 0-4 wt. %, or 0-3 wt. %,
F: 10-18 wt. %, or 11-16 wt. %,
O: 25-35 wt. %, or 28-34 wt. %,
Si: 10-16 wt. %, or 11-14 wt. %,
Al: 11-19 wt. %, or 12-18 wt. %,
Sr: 20-40 wt. %, or 20-38 wt. %,
La: 0-4 wt. %, or 0-3 wt. %,
the combined amounts of Al, Sr and F being >48 wt. %, wt. % with respect to the weight of the acid-reactive glass, and the ratio of Al and Si in the acid-reactive glass being greater than 1/1 with respect to weight.

In another embodiment, the invention relates to a glass ionomer composition obtainable from the kit of parts as described in the present text and claims.

The invention also relates to a kit of parts comprising in addition the following items alone or in combination: activating device, application device, mixing device, dental milling block, preformed dental restoration.

The invention is also directed to the use of the acid reactive glass and the polycarboxylic acid as described in the present text in combination for improving the mechanical strength of a glass ionomer composition.

A further embodiment of the invention is directed to glass ionomer composition for use in a method of treating a dental defect in the mouth of a patient as described in the present text and claims.

Unless defined differently, for this description the following terms shall have the given meaning:

The term “compound” or “component” is a chemical substance which has a certain molecular identity or is made of a mixture of such substances, e.g., polymeric substances.

A “hardenable or curable or polymerizable component” is any component which can be cured or solidified e.g. in the presence of a photo-initiator by radiation-induced polymerization or by a glass-ionomer reaction, that is a reaction between a polyacid and an acid-reactive filler. A polymerizable component may contain only one, two, three or more polymerizable groups. Typical examples of polymerizable groups include unsaturated carbon groups, such as a vinyl group being present i.a. in a (methyl) acrylate group.

As used herein, “(meth)acryl” is a shorthand term referring to “acryl” and/or “methacryl”. For example, a “(meth)acryloxy” group is a shorthand term referring to either an acryloxy group (i.e., CH₂═CH—C(O)—O—) and/or a methacryloxy group (i.e., CH₂═C(CH₃)—C(O)—O—).

As used herein, “hardening” or “curing” a composition are used interchangeably and refer to polymerization and/or crosslinking reactions including, for example, photo-polymerization reactions and chemical-polymerization techniques (e. g., ionic reactions or chemical reactions forming radicals effective to polymerize ethylenically unsaturated compounds) involving one or more materials included in the composition.

An “initiator” is a substance being able to start or initiate the curing process of radically polymerizable components or monomers, e.g. redox/auto-cure chemical reaction or by a radiation induced reaction or by a heat induced reaction.

“Dental restoration” means dental articles which are used for restoring a tooth to be treated. Examples of dental restorations include fillings, crowns, bridges, inlays, onlays, veneers, facings, copings, crown and bridged framework, and parts thereof.

A “particle” means a substance being a solid having a shape which can be geometrically determined. The shape can be regular or irregular. Particles can typically be analysed with respect to e.g. particle size and particle size distribution.

The particle size (d50) of a powder can be obtained from the cumulative curve of the grain size distribution. Respective measurements can be done using commercially available granulometers (e.g. Malvern Mastersizer 3000). “D” represents the diameter of powder particles and “50” refers to the volume percentage of the particles. Sometimes, the 50% is also expressed as “0.5”. For example, “(d50)=1 μm” means that 50% of the particles have a size of 1 μm or less.

A “powder” means a dry, bulk solid composed of a large number of fine particles that may flow freely when shaken or tilted.

“Glass ionomer cement” or “GIC” shall mean a cement curing or hardening by the reaction between an acid-reactive glass and a polycarboxylic acid in the presence of water.

“Resin modified glass ionomer cement” or “RM-GIC” shall mean a GIC containing in addition radically polymerizable component(s), an initiator system and typically 2-hydroxyl-ethyl-methacrylate (HEMA).

The kit of parts described in the present text relates to a glass ionomer cement, but not to a resin modified glass ionomer cement.

“Acid-reactive filler or glass” shall mean a filler or glass that chemically reacts in the presence of an acidic component.

“Non acid-reactive filler” shall mean a filler, which does not show a chemical reaction within 6 min at all, if mixed with a (poly)acid or which shows only a reduced (i.e. time-delayed) reaction.

To distinguish an acid-reactive filler from a non acid-reactive filler the following test can or is to be conducted:
A composition is prepared by mixing Part P with Part L in a mass ratio of 3 to 1, wherein:
Part P contains: filler to be analysed: 100 wt. %.

Part L contains: poly (acrylic acid co maleic acid) (Mw: about 18,000+/−3,000): 43.6 wt. %, water: 47.2 wt. %, tartaric acid: 9.1 wt. %, benzoic acid: 0.1 wt. %.

The filler is characterized as non-acid reactive, if within 6 min after preparing the above composition the shear stress is less than 50,000 Pa, if determined by conducting an oscillating measurement using a rheometer by applying the following conditions: using an 8 mm plate, 0.75 mm gap, at 28° C., frequency: 1.25 Hz, deformation: 1.75%.

“Cation reduced aluminosilicate glasses” shall mean a glass having a lower content of cations in the surface region of the glass particle compared to the inner region of the glass particle.

These glasses react much slower upon contact with a solution of polyacrylic acid in water as compared to typical acid-reactive fillers. Examples of non acid-reactive fillers include quartz glass. Further examples are given in the text below.

Cation reduction can be achieved by a surface treatment of the glass particles. Suitable surface treatments include, but are not limited to, acid washing (e.g., treatment with a phosphoric acid or with hydrochloric acid), treatment with a phosphate or treatment with a chelating agent such as tartaric acid.

“Polycarboxylic acid or polycarboxylic acid or polyalkenoic acid” shall mean a polymer having a plurality of acidic repeating units (e.g. more than 10 or more than 20 or more than 50). That is, the acidic repeating units are attached to or pending from the backbone of the polymer.

“Complexing agent” or “chelating agent” shall mean a low molecular agent comprising moieties and being able to form a complex with metal ions like calcium or magnesium; e.g. tartaric acid. The terms “complexing agent” and “chelating agent” are interchangeable.

A “storage stable composition” is a composition which can be stored for an adequate period of time (e.g. at least about 12 months under ambient conditions) without showing significant performance issues (e.g. reduced flexural or compressive strength and/or which does not harden in the desired period of time (e.g. setting time greater than 6 min)) when used. A suitable test for determining the storage stability is given in the Example section below.

“Ambient conditions” mean the conditions which the composition described in the present text is usually subjected to during storage and handling. Ambient conditions may, for example, be a pressure of 900 to 1,100 mbar, a temperature of 10 to 40° C. and a relative humidity of 10 to 100%. In the laboratory ambient conditions are typically adjusted to 20 to 25° C. and 1,000 to 1,025 mbar (at maritime level).

As used herein, “a”, “an”, “the”, “at least one” and “one or more” are used interchangeably. Also herein, the recitations of numerical ranges by endpoints include all numbers subsumed within that range (e.g., 1 to 5 includes 1, 1.5, 2, 2.75, 3, 3.80, 4, 5, etc.).

Adding an “(s)” to a term means that the term should include the singular and plural form. E.g. the term “additive(s)” means one additive and more additives (e.g. 2, 3, 4, etc.).

Unless otherwise indicated, all numbers expressing quantities of ingredients, measurement of physical properties such as described below and so forth used in the specification and claims are to be understood as being modified in all instances by the term “about”.

The terms “comprise” or “contain” and variations thereof do not have a limiting meaning where these terms appear in the description and claims. “Consisting essentially of” means that specific further components can be present, namely those which do not materially affect the essential characteristic of the article or composition. “Consisting of” means that no further components should be present. The term “comprise” shall include also the terms “consist essentially of” and “consists of”.

A composition is “essentially or substantially free of” a certain component, if the composition does not contain said component as an essential feature. Thus, said component is not wilfully added to the composition either as such or in combination with other components or ingredient of other components. A composition being essentially free of a certain component usually does not contain that component at all. However, sometimes the presence of a small amount of the said component is not avoidable e.g. due to impurities contained in the raw materials used (e.g. less than 1 wt. % or less than 0.5 wt. % or less than 0.1 wt. % or less than 0.01 wt. % with respect to the whole composition or material).

### DETAILED DESCRIPTION

It has been found that the kit of parts and glass ionomer composition described in the text has a couple of advantageous properties.

The hardened glass ionomer composition shows advantageous mechanical strength properties, like high compressive strength and/or surface hardness.

In particular the glass ionomer composition shows a sufficient surface hardness after a short period of time. This enables the practitioner to further trim the surface and/or shape of the filling already shortly after the application of the material, if desired.

Further, the components of the powder part and the liquid part of the kit of parts can easily be mixed and the respective components, in particular the polycarboxylic acid and the acid-reactive glass show a smooth hardening reaction.

It has been found that the combination of a particular polycarboxylic acid with a particular acid-reactive glass contributes to an improvement of mechanical properties.

An acid-reactive glass with the claimed Al/Si ratio and a comparably high content of Al, Sr and F in combination shows a reactivity which matches very well with the reactivity of a polycarboxylic acid comprising a copolymer of acrylic acid and maleic acid.

If desired, the particle size distribution of the non-acid reactive glass can be adjusted to even further improve the mechanical properties.

The invention relates to a kit of parts for obtaining a glass ionomer composition. The kit of parts comprises or essentially consists of or consists of a Part P and a Part L.

Part P is a powder component or composition. Part L is a liquid component or composition.

Part L can typically be characterized by a viscosity in the range of 1 to 500 Pa*s or 1 to 100 Pa*s or 1 to 50 Pa*s or 1 to 10 Pa*s (28° C.; 10 mm diameter; shear rate: 1 s⁻¹), wherein a viscosity in the range of 1 to 50 Pa*s or 1 to 10 Pa*s is often preferred; density: 1.1 to 2.0 g/cm³.

According to one embodiment, Part L comprises water, polycarboxylic acid and optionally a complexing or chelating agent.

For obtaining a glass ionomer composition, the parts of the kit of parts described in the present text need to be mixed.

An appropriate mixing ratio is typically within a range of 6:1 to 1:1 with respect to weight. A mixing ratio of 4:1 to 1:1 with respect to weight is sometimes preferred.

Part P of the kit of parts comprises an acid-reactive glass.

The following particle size distribution of the acid-reactive glass was found to be useful: 15 μm (d90) and 2 μm (d50). That is, 90% of the particles have a size of 15 μm or smaller and 50% of the particles have a size of 2 μm or smaller. This means that 10% of the particles still have a size of greater than 15 μm.

Without wishing to be bound to a particular theory, it is believed that such a particle distribution helps to increase the packaging density of the glass particles in the cured composition and contributes to increasing the compressive strength.

Alternatively, the following particle size distribution was found to be useful: 10 μm (d90) and 1 μm (d50).

The composition of the acid-reactive glass is as follows: P: 0-4 wt. %, F: 10-18 wt. %, O: 25-35 wt. %, Si: 10-16 wt. %, Al: 11-19 wt. %, Sr: 20-40 wt. %, La: 0-4 wt. %, wherein the combined amount of Al, Sr and F is greater than 48 wt. % or greater than 50 wt. %.

Alternatively, the following acid-reactive glass composition can be used: P: 0-3 wt. %, F: 11-16 wt. %, O: 28-34 wt. %, Si: 11-14 wt. %, Al: 12-18 wt. %, Sr: 20-38 wt. %, La: 0-3 wt. %, wherein the combined amount of Al, Sr and F is greater than 48 wt. % or greater than 50 wt. %.

The acid-reactive glass can be produced by melting a glass frit containing the respective glass components, crushing and grinding the glass frit until the desired particle size distribution is obtained.

Glass components which can be used include Al₂O₃, SiO₂, SrF₂ and AlF₃-hydrate or AlF₃.

The milling or grinding of the glass frit can be done e.g. with a ball mill.

The Al/Si ratio of the acid-reactive glass of the invention is greater than 1/1 with respect to weight. This means that the acid-reactive glass contains more Al than Si. An Al/Si ratio in the range of greater than 1.0/1.0 to 1.6/1.0 or greater than 1.0/1.0 to 1.4/1.0 with respect to weight is often preferred.

Such a ratio was found to be useful as it has an impact on the reactivity of the glass with respect to the polycarboxylic acid.

The acid-reactive glass does typically not comprise the following elements alone or in combination: Li, K, Rb, Cs, Be, Mg, each in an amount of more than 0.2 wt. % with respect to the weight of the acid-reactive glass.

For adjusting its reactivity, the acid-reactive glass can be deactivated by treating the glass powder with acid, in particular acid having a pKs of <3, e.g. with hydrochloric acid, followed by washing with water and drying.

A glass powder treated by such a process is less reactive. By using a less reactive glass powder, the hardening reaction with the polycarboxylic acid proceeds slower and in a more controlled way, which may further contribute to the mechanical properties of the hardened glass ionomer composition.

A glass obtained by such a process is often referred to as cation reduced aluminosilicate glass.

The acid-reactive glass is typically present in the following amounts: at least 25 or at least 35 or at least 45 wt. %; at most 86 or at most 83 or at most 80 wt. %; or in a range of: 25 to 86 or 35 to 83 or 45 to 80 wt. %, wt. % with respect to the weight of the composition obtained when combining Part P and Part L.

If the amount of the acid-reactive inorganic filler is too low, a suitable paste cannot be obtained by mixing the respective parts of the kit of parts described in the present text. Further, the mechanical properties might become inferior.

The kit of parts described in the present text also comprises a liquid Part L.

One component of Part L is water.

Water is typically present in the following amounts: at least 2 or at least 5 or at least 7 wt. %; at most 35 or at most 25 or at most 20 wt. %; or in a range of: 2 to 35 or 5 to 25 or 7 to 20 wt. %; wt. % with respect to the weight of the composition obtained when combining Part P and Part L.

These amounts were found to be useful for obtaining a glass ionomer composition having adequate mechanical properties after hardening.

The kit of parts also comprises polycarboxylic acid. The polycarboxylic acid can be present in Part P or Part L or Part P and Part L.

If the polycarboxylic acid is present in Part P, the polycarboxylic acid is present in dry form.

A dry polycarboxylic acid can be obtained e.g. by spray drying of an aqueous solution of the polycarboxylic acid (e.g. 10 wt. %) in a spray dryer under vacuum.

According to the invention, the polycarboxylic acid comprises or essentially consists of or consists of a copolymer of acrylic acid and maleic acid.

The polycarboxylic acid should have a molecular weight sufficient to provide good storage, handling, and mixing properties, as well as to yield good material properties in the glass ionomer material.

According to one embodiment, the polycarboxylic acid can be characterized by the following properties alone or in combination:
being a solid (at 23° C.);
   molecular weight (Mw): 5,000 to 250,000 g/mol or 10,000 to 100,000 g/mol (evaluated against a polyacrylic acid sodium salt standard using gel permeation chromatography).

If the molecular weight of the polycarboxylic acid is too high, obtaining a workable consistency of the obtained paste when mixing the compositions contained in the kit of parts described in the present text might become difficult. Further, preparation of the compositions might become difficult. In addition, the obtained mixture or composition might become too sticky (i.e. adheres to a dental instrument used for application).

If the molecular weight of the polycarboxylic acid is too low, the viscosity of the obtained paste is supposed to become too low and the mechanical properties of the final product inferior. The polycarboxylic acid is a polymer having a plurality of acidic repeating units.

The polycarboxylic acid to be used for the glass ionomer composition described in the present text is substantially free of polymerizable groups.

The polycarboxylic acid does not need to be entirely water soluble, but typically it is at least sufficiently water-miscible so that it does not undergo substantial sedimentation when combined with other aqueous components.

The polycarboxylic acid is hardenable in the presence of an acid-reactive filler and water but does not contain ethylenically unsaturated groups.

That is, the polycarboxylic acid is a polymer obtained by polymerising an unsaturated acid. However, due to the production process, a polycarboxylic acid might still contain unavoidable traces of free monomers (e.g. up to 1 or up to 0.5 or up to 0.3 wt. % with respect to the amount of monomers used).

The polycarboxylic acid typically contains acrylic acid and maleic acid in the following molar ratio: 35-65% maleic acid to 65-35% acrylic acid, or 40-60% maleic acid to 60-40% acrylic acid.

A polycarboxylic acid with such a ratio was found to be in particular suitable for obtaining a glass ionomer composition having high compressive strength if reacted with the acid-reactive glass described in the present text.

The amount of polycarboxylic acid to be used should be sufficient to react with the acid-reactive filler and to provide an ionomer composition with desirable hardening properties.

The polycarboxylic acid is typically present in the following amount: at least 3 or at least 6 or at least 8 wt. %; at most 35 or at most 25 or at most 20 wt. %; or in a range of: 3 to 35 or 6 to 25 or 8 to 20 wt. %; wt. % with respect to the weight of the composition obtained when combining Part P and Part L.

If the amount of the polycarboxylic acid is too high, obtaining a workable consistency of the obtained paste when mixing the compositions contained in the kit of parts described in the present text might become difficult. Further, preparation of the compositions might become difficult. In addition, the obtained mixture or composition might become too sticky (i.e. adheres to the dental instrument used for application).

If the amount of the polycarboxylic acid is too low, obtaining a workable consistency of the obtained paste when mixing the compositions contained in the kit of parts described in the present text might become difficult, either. Further, it will become difficult to achieve the desired mechanical properties.

The kit of parts may further comprise a complexing agent. A complexing agent is often used for adjusting the setting properties of the glass ionomer composition.

If present, the complexing agent can be present in Part P or Part L or Part P and Part L.

The nature and structure of the complexing agent is not particularly limited unless the desired result cannot be achieved.

The complexing agent can be characterized by the following properties alone or in combination: solubility: soluble in water (at least 50 g/l water at 23° C.); molecular weight: 50 to 500 g/mol, or 75 to 300 g/mol.

Specific examples of the complexing agent include tartaric acid, citric acid, ethylene diamine tetra acetic acid (EDTA), salicylic acid, mellitic acid, dihydroxy tartaric acid, nitrilotriacetic acid (NTA), 2,4 and 2,6 dihydroxybenzoic acid, phosphono carboxylic acids, phosphono succinic acid and mixtures thereof. The use of tartaric acid is often preferred.

Further examples can be found e.g. in U.S. Pat. No. 4,569,954 (Wilson et al.).

The complexing agent is typically added to that part containing the polycarboxylic acid. Typically, the complexing agent is present in Part L.

The complexing agent is typically present in the following amounts: at least 0 or at least 1 or at least 2 wt. %; at most 15 or at most 12 or at most 10 wt. %; or in a range of: 0 to 15 or 1 to 12 or 2 to 10 wt. %; wt. % with respect to the weight of the composition obtained when combining Part P and Part L.

Part P of the kit of parts described in the present text can also contain other non-acid reactive filler(s).

The non acid-reactive filler may have a particle size of (d10): 0.2 μm to 2 μm; (d50): 0.5 μm to 5 μm; (d90) 1 μm to 15 μm.

Examples of suitable non-acid reactive fillers are naturally occurring or synthetic materials including, but not limited to: quartz; nitrides (e.g., silicon nitride); glasses derived from, e.g., Zr, Sr, Ce, Sb, Sn, Ba, Zn, and Al; borosilicate glass; kaolin; silica particles (e.g. quartz glass or pyrogenic silica of suitable particle size), alumina, titania and zirconia particles.

According to one embodiment, the non-acid reactive filler is selected from quartz, quartz glass, silica, alumina, aluminosilicates and mixtures thereof.

If desired, the surface of the particles of the acid-reactive filler can be surface treated.

Conducting a surface treatment can be beneficial for improving the compatibility of the filler with the other components of the glass ionomer composition.

Suitable surface-treating agents include silanes, e.g. trimethoxysilanes carrying an organic functional group to modify the chemical properties of the particles. Suitable silanes are e.g. silanes to modify the acidic properties (carrying amino groups or carrying carboxylic acid groups) or silanes to modify the hydrophobicity/hydrophilicity (carrying an alkane chain or carrying a polyethylene glycol chain).

The non acid-reactive filler is typically present in the following amounts: 0 or at least 1 or at least 2 wt. %; at most 35 or at most 30 or at most 25 wt. %; or in a range of: 0 to 35 or 1 to 30 or 2 to 25 wt. %; wt. % with respect to the weight of the composition obtained when combining Part P and Part L.

Either Part P or Part L or Part P and Part L of the kit of parts described in the present text can also contain additive(s).

Additives which might be present include indicator(s), dye(s), pigment(s), viscosity modifier(s), surfactant(s), buffering agent(s), stabilizer(s), preservative agent(s) (e.g., benzoic acid).

If additives are present, Part P may contain additive(s) which can be provided in powder form.

Combination of any of the above additives may also be employed. The selection and amount of any one such additive can be selected by one of skill in the art to accomplish the desired result without undue experimentation.

Additive(s) may be present in the following amounts: at least: 0 or 0.1 or 0.2 wt. %; at most: 10 or 8 or 6 wt. %; or in a range of: 0 to 10 or 0.1 to 8 or 0.2 to 6 wt. %; wt. % with respect to the weight of the composition obtained when combining Part P and Part L.

According to one embodiment, the kit of parts comprises, consists essentially of or consists of the following components in the following amounts:
acid-reactive glass: 25 to 86 wt. %,
non acid-reactive filler: 0 to 35 wt. %,
polycarboxylic acid: 3 to 35 wt. %,
water: 2 to 35 wt. %,
complexing agent: 0 to 15 wt. %,
additives: 0 to 10 wt. %,
wt. % with respect to the weight of the composition obtained when combining Part P and Part L.

According to another embodiment, the kit of parts comprises, consists essentially of or consists of the following components in the following amounts:
acid-reactive glass: 35 to 83 wt. %,
non acid-reactive filler: 1 to 30 wt. %,
polycarboxylic acid: 6 to 25 wt. %,
water: 5 to 25 wt. %,
complexing agent: 1 to 12 wt. %,
additives: 0 to 8 wt. %,
wt. % with respect to the weight of the composition obtained when combining Part P and Part L.

A preferred embodiment of the kit of parts can be characterized as follows:
consisting of Part P and Part L,
Part P being a powder comprising acid-reactive glass,
Part L being a liquid comprising water, polycarboxylic acid, and complexing agent,
the polycarboxylic acid comprising a copolymer of acrylic acid and maleic acid,
the acid-reactive glass being characterized by comprising
   P: 0-4 wt. %, or 0-3 wt. %,
   F: 10-18 wt. %, or 11-16 wt. %,
   O: 25-35 wt. %, or 28-34 wt. %,
   Si: 10-16 wt. %, or 11-14 wt. %,
   Al: 11-19 wt. %, or 12-18 wt. %,
   Sr: 20-40 wt. %, or 20-38 wt. %,
   La: 0-4 wt. %, or 0-3 wt. %,
      the combined amount of Al, Sr and F being >48 wt. %, wt. % with respect to the weight of the acid-reactive glass,
the ratio of Al and Si in the acid-reactive glass being greater than 1/1 with respect to weight,
the acid-reactive glass having a particle size distribution (d90) of 10 μm and 2 μm (d50),
the acid-reactive glass having been deactivated by treating it with acid, followed by washing with water and drying,
the polycarboxylic acid having a molecular weight Mw in the range of 10,000 to 100,000 g/mol,
the acrylic acid and maleic acid being present in the polycarboxylic acid in a molar ratio of 35-65% maleic acid and 65-35% acrylic acid, or 40-60% maleic acid to 60-40% acrylic acid,
the complexing agent being tartaric acid,
Part P comprising in addition a non acid-reactive glass selected from aluminosilicates.

Typically, neither Part P nor Part L of the kit of parts described in the present text contains the following components alone or in combination:
a) HEMA in an amount above 1 wt. % or above 0.5 wt. %;
b) radically polymerizable component(s) in an amount above 1 wt. % or above 0.5 wt. %;
c) initiator component(s) suitable to cure radically polymerizable component(s) or monomer(s) in an amount above 1 wt. % or above 0.5 wt. %;
d) inhibitor(s) like methoxyphenol or 3,5-Di-tert-butyl-4-hydroxytoluol in an amount above 1 wt. % or above 0.5 wt. %
   wt. % with respect to the weight of the composition obtained when combining Part P and Part L.

Thus, the composition obtained when mixing the powder and liquid part of the kit of parts described in the present text is not a so-called resin-modified glass ionomer cement (RM-GIC) and thus does not contain a curing system suitable for curing radically polymerizable components.

In particular, the cement composition described in the present text does not contain a redox-initiator system or a thermally induced initiator system or a radiation induced initiator system.

In particular the cement composition described in the present text does not contain the following components: (a) and (b), (a) and (c), (a), (b) and (c), (b), (c) and (d), (a), (b), (c) and (d) in an amount of 1 wt. % or more, or 0.5 wt. % or more, or 0.1 wt. % or more with respect to the weight of the whole composition.

That is, the cement composition described in the present text is typically essentially free of these components alone or in combination.

The composition obtained or obtainable by mixing the two parts of the kit of parts described in the present text typically can be characterized by the following parameters alone or in combination before or during hardening:
Setting time: within about 5 or 4 or 3 min determined according to EN-ISO 9917-1:2007;
Working time: within about 4 or 3 or 2 or 1 min determined according to EN-ISO 9917-1:2007;
Viscosity: 2,000 to 10,000 Pa*s at 28° C. measured 90 sec after start of mixing the components of Part P and Part L.

If desired, the setting time and curing behaviour can be determined as described in more detail in the example section below.

The composition described in the present text typically has a sufficient working time allowing the practitioner not only to adequately mix the composition but also to apply the composition to a cavity or the surface of a crown, bridge, root canal or prepared tooth.

Further, the composition described in the present text has an adequate setting time, which is time saving for the practitioner and convenient for the patient.

According to another embodiment the composition obtained or obtainable by mixing the two parts of the kit of parts described in the present text can be characterized by the following parameters alone or in combination after hardening:
flexural strength: 20 MPa to 80 MPa determined according to EN-ISO 9917-2:2010 with the proviso that for covering the composition a glass slab is used instead of a foil;
compressive strength: 300 MPa to 400 determined according to EN-ISO 9917-1/2007 with the proviso that for covering the composition a glass slab is used instead of a foil;
surface hardness: 150 to 250 MPa.

If desired, these parameters can be determined as described in the example section below.

Compared to state of the art glass ionomer compositions available on the market, the glass ionomer composition described in the present text can easily be mixed and has adequate mechanical properties like compressive and/or flexural strength without affecting other important parameters like setting time.

Further, the composition shows a sufficient surface hardness already 10 min after mixing.

The parts of the kit of parts of the present text can be produced by mixing the respective components.

If needed, filler particles can be milled to the desired particle size using equipment known to the skilled person like ball mills.

Mixing can be accomplished either by hand or with a mechanical device like a mixer or kneading machine. The mixing duration can vary depending on the composition and the mixing device.

The kit of parts is typically contained in a packaging device, during storage and before use.

The powder of Part P and/or the liquid of Part L can be stored in any suitable device separated from each other before use, such as a vessel, vial or cartridge.

A preferred packaging device comprises at least two compartments suitable for storing the liquid and the powder part.

A suitable packaging device can be characterized as follows:

Device for storing and delivery of the kit of parts described in the present text, the device comprising Compartment A and Compartment B separated from each other during storage and a nozzle connected to either Compartment A or Compartment B, Compartment A containing Part P and Compartment B containing Part L, wherein Compartment A has a volume in the range of 0.5 to 3 ml or 0.8 to 2 ml and Compartment B has a volume in the range of 0.05 to 1 ml or 0.08 to 0.5 ml.

Packaging devices which can also be used are described in the following documents: U.S. Pat. No. 6,543,611 B1 (3M), U.S. Pat. No. 4,941,751 (Muehlbauer), U.S. Pat. No. 5,088,830 (Muehlbauer), U.S. Pat. No. 6,386,872 (Muasa et al.) or EP 0 783 872 A2 (Voco).

The kit of parts can and typically is to be used for treating a tooth, in particular a tooth located in the mouth of a patient.

Treating a tooth includes restoring a tooth, e.g. by filling a cavity in the tooth, fixing a dental restoration to a tooth surface.

Thus, the kit of parts can be used for producing a dental luting cement, dental filling material, dental core build up material, dental liner or as dental root channel filling material.

The process of treating a tooth typically comprises the steps
mixing the components or compositions of Part P and Part L to obtain a hardenable composition,
applying the hardenable composition to the surface of dental tissue to be treated,
letting the hardening composition harden.

The invention also relates to a kit of parts comprising the kit of parts described in the present text and at least one or more of the following items: activating device; application device; mixing device; dental milling block; preformed dental restorations (including dental crowns or bridges).

Suitable activating devices are commercially available, e.g. 3M™ Maxicap™ and Aplicap™ Activator.

Application devices are often used for extruding the mixed composition from the packaging device and applying the mixed composition to the surface to be treated.

Mixing devices are used for mixing the powder and liquid part. Suitable mixing devices include a mixing pad and a spatula (in particular useful for mixing the parts by hand) or electrical shaking or rotating mixing devices. Such products are commercially available e.g. 3M™ RotoMix™ Capsule Mixing Unit.

Dental milling blocks can be used for milling dental restorations therefrom which are later fixed to a tooth surface to be treated. Dental milling blocks are often made from zirconia and are also commercially available, e.g. 3M™ Lava™ Plus zirconia disc.

Alternatively, preformed dental restorations can be used, including polycarbonate crowns and stainless steel crowns (3M Oral Care).

All components used in the composition of the present text should be sufficiently biocompatible, that is, the composition should not produce a toxic, injurious, or immunological response in living tissue.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. The above specification, examples and data provide a description of the manufacture and use of the compositions and methods of the invention. The invention is not limited to the embodiments disclosed herein. One skilled in the art will appreciate that many alternative embodiments of the invention can be made without departing from the spirit and scope of thereof.

The following examples are given to illustrate the invention.

### EXAMPLES

Unless otherwise indicated, all parts and percentages are on a weight basis, all water is de-ionized water, and all molecular weights are weight average molecular weight. Moreover, unless otherwise indicated all experiments were conducted at ambient conditions (23° C.; 1013 mbar).

### Methods

### Viscosity

If desired, viscosity can be measured using a Physica MCR 301 Rheometer (Anton Paar, Graz, Austria) with a plate/plate geometry under controlled shear rate at 23° C. The diameter is 15 mm, the separation gap between the plates 0.5 mm. The shear rate is ramped from 1 s⁻¹ to 500 s⁻¹.

### Particle Size

If desired, the particle size distribution including the particle size (d50) per volume can be determined by laser diffraction with a Mastersizer 3000 (Malvern) particle size detection device applying the Fraunhofer approximation. During the measurement, ultrasonic is typically used to accurately disperse the sample. For water-insoluble particles, water is typically used as dispersant.

### pH Value

If desired, the pH value of can be determined as follows: 1.0 g of a component (e.g. filler) is dispersed in 10 ml de-ionized water and stirred for about 5 min. A calibrated pH electrode is dipped into the suspension and the pH value is determined during stirring.

### Elemental Composition

If desired, the elemental composition can be determined by X-ray fluorescence spectrometry (XRF), e.g. with the ZSX Primus II from Rigaku, Japan. This method is especially suited for the analysis of solids, e.g. zirconia ceramics or glass materials.

### Compressive Strength (CS)

Measurement of the compressive strength was carried out according to the EN-ISO 9917-1:2007 with the proviso that for covering the composition a glass slab is used instead of a foil. Cylindrical specimens with a diameter of 4 mm and a height of 6 mm were used. Specimens of the materials were prepared at room temperature and 50% relative humidity using split moulds. The moulds were placed on microscope slides and thoroughly filled with the mixed material to avoid incorporation of air bubbles. The filled moulds were immediately covered with another glass slab and fixed in a screw clamp with slight pressure to extrude excess material. The whole assembly was stored at 36° C. and at least 95% relative humidity. 1 h after start of mixing the specimens were removed from the moulds and immediately placed in water at 36° C. 6 specimens were prepared for each material. Materials were measured 24 h after start of mixing. The exact diameter of each specimen was measured prior to the measurement. The strength of the specimen was measured by applying a compressive load using a Zwick universal testing machine (Zwick GmbH & Co. KG, Ulm, Germany) operating at a crosshead speed of 1 mm/min. Results were reported as an average of 6 replications.

### Flexural Strength (FS)

Flexural strength was measured based on EN ISO 9917-2:2010 with the proviso that for covering the composition a glass slab is used instead of a foil. The specimens were prepared as described for the compressive strength test above, except that rectangular-shaped split moulds with dimensions 25 mm×2 mm×2 mm were used to prepare the samples. The specimens were subjected to a 3-point bend on supports 20 mm apart at a crosshead speed of 1 mm/min.

Working Time (ta) and Setting Time (the)

If desired, the setting behaviour of the prepared glass ionomer cement composition can be determined using a Physica™ MCR 301 Rheometer (Anton Paar) applying the following parameters: Oscillating measurement with 8 mm disc on disc set-up; gap 0.75 mm; deformation 1.75%; frequency: 1.25 HZ; temperature: 28° C. The loss angle (in German: “Verlustwinkel”) is recorded over time and the maximum (ta) and the minimum (the) of the graph determined. The average of two measurements with respect to the maximum and the minimum is given in min: sec.

### Surface Hardness (SFH)

If desired, the surface hardness (given in MPa) is determined with a steel ball (d=5 mm) according to ISO 2039-1. The sample material is filled into an aluminum ring (inner diameter 6 mm±0.2 mm, height 3 mm±0.1 mm) and closed on both sides with a plastic plate and fixed with a clamp. 3 min after the start of mixing, the sample is placed for 2 min in a water bath with 36° C.±2° C., then for 5 min in a water bath with 23° C.±2° C. The removal of the plastic plate takes place 10 min after the start of mixing, the measurement of the penetration depth takes place 10.5 min after the start of mixing for 30 s. For the measurement, a hardness tester type 3106 (Zwick) can be used.

### Acid Reactive Glass

### General Preparation

The glass components are weighed and homogenized. The mixture is pre-tempered in a crucible. After that the cooled mixture is transferred to a platinum crucible and heated to a temperature above 1,500° C. The melt is removed and poured directly into de-ionised water. The cooled melt is then dried and ground in a planetary ball mill until the desired particle size distribution is achieved. The glass powder is treated with an acid, washed and dried.

The glass powder compositions produced are shown in Table 1.

TABLE 1

| Element | | | | | | | | | |
|---|---|---|---|---|---|---|---|---|---|
| (wt. %) | ARG1 | ARG2 | ARG3 | ARG4 | ARG5 | ARG6 | ARG7 | ARG8 | ARG9 |
| O | 27.66 | 31.80 | 33.09 | 32.41 | 29.04 | 32.03 | 30.92 | 32.03 | 31.59 |
| F | 11.32 | 13.66 | 13.85 | 14.51 | 14.67 | 13.04 | 14.40 | 13.04 | 12.47 |
| Na | 0.90 | 2.40 | 1.17 | 0.24 | 0.09 | 1.32 | 0.19 | 1.32 | 2.56 |
| Al | 11.82 | 14.94 | 15.83 | 16.15 | 17.14 | 15.57 | 16.64 | 15.57 | 14.19 |
| Si | 11.02 | 12.99 | 15.26 | 13.09 | 11.22 | 13.04 | 13.46 | 13.04 | 12.47 |
| P | 0.91 | 1.21 | 0.02 | 0.11 | 0.05 | 1.57 | 0.02 | 1.57 | 2.15 |
| Sr | 36.37 | 23.00 | 20.78 | 23.49 | 27.79 | 23.43 | 24.37 | 23.43 | 18.81 |
| La | | | | | | | | | 5.76 |
| Al/Si | 1.07 | 1.15 | 1.04 | 1.23 | 1.53 | 1.19 | 1.24 | 1.19 | 1.14 |
| ratio | | | | | | | | | |
| Al + Sr + F | 59.51 | 51.60 | 50.46 | 54.15 | 59.60 | 52.04 | 55.41 | 52.04 | 45.47 |

ARG1-8 are acid-reactive glasses according to the invention. ARG9 is a comparative glass.

### Polycarboxylic Acid (PA)

Different polycarboxylic acids were prepared (Table 2).

TABLE 2

| # | Description | Mw(g/mol) |
|---|---|---|
| PA1 | Copolymer of acrylic acid and maleic acid in a | 20.000 |
| | molar ratio of 40:60 to 60:40 | |
| PA2 | Copolymer of acrylic acid and itaconic acid in a | |
| | molar ratio of 45:55 to 55:45 | |
| PA3 | Copolymer of acrylic acid and maleic acid in a | |
| | molar ratio of 65:35 to 70:30 | |
| PA 4 | polyacrylic acid | 60.000 |

The following liquid parts were produced:

### Part L (Liquid)

TABLE 3

| Part L | L1 | L2 | L3 | L4 |
|---|---|---|---|---|
| PA | PA1 | PA2 | PA3 | PA4 |
| Acid | 48.2% | 49.0% | 47.6% | 38.2% |
| Tartaric acid | 9.1% | 9.1% | 9.1% | 9.1% |
| Water | 42.7% | 41.9% | 43.3% | 52.7% |
| Viscosity at 1 s⁻¹ | 1.2 Pa*s | 2.6 Pa*s | 2.2 Pa*s | — |

### Glass Ionomer Compositions (GIC)

The respective powder parts and liquid parts were weight into a capsule and mixed by a CapMix™ device (3M Oral Care) in the given ratio with respect to weight (pbw-parts by weight). The properties of the hardened compositions are given in Table 5.

TABLE 5

| GIC | GIC1 | GIC2 | GIC3 | GIC4 | GIC5 | GIC6 | GIC7 | GIC8 |
|---|---|---|---|---|---|---|---|---|
| Part P (pbw) | 3.3 | 3.1 | 2.8 | 3.4 | 3.4 | 3.4 | 3.3 | 3.4 |
| Part L (pbw) | 1.0 | 1.0 | 1.0 | 1.0 | 1.0 | 1.0 | 1.0 | 1.0 |
| Powder | ARG 1 | ARG 2 | ARG 3 | ARG 8 | ARG 8 | ARG 8 | ARG 9 | ARG 8 |
| Liquid | L1 | L1 | L1 | L1 | L2 | L3 | L1 | L4 |
| CS (MPa) | 316.4 | 318.3 | 312.6 | 311.1 | 198.4 | 257.5 | 279.5 | 157.2 |
| SFH (MPa) | 158.9 | 160.3 | 182.3 | — | — | — | — | — |

For further comparison the following commercially available materials were tested (Table 6):

TABLE 6

| Manufacturer | GC | GC | GC | Voco | Densply | SDI |
|---|---|---|---|---|---|---|
| Product | Fuji ™ IX | Equia ™ | Equia ™ | Ionostar ™ | Chemfil ™ | Riva ™ sc |
| | | Forte | Forte HT | Plus | Rock | reg, Set |
| CS (MPa) | 204.2 | 242.9 | 198.1 | 205.5 | 182.8 | 203.1 |
| SFH (MPa) | 63.0 | 60.5 | 61.8 | 49.1 | 37.6 | 69.4 |

The glass ionomer cement compositions according to the invention showed improved mechanical properties, in particular with respect to compressive strength and surface hardness over the glass ionomer cement compositions according to the state of the art.

1. A kit of parts for obtaining a glass ionomer composition, the kit of parts comprising Part P and Part L,
Part P being a powder comprising acid-reactive glass,
Part L being a liquid comprising water,
the kit of parts comprising in addition a polycarboxylic acid, the polycarboxylic acid being present in Part P or Part L or Part P and Part L, the polycarboxylic acid comprising a copolymer of acrylic acid and maleic acid,
the acid-reactive glass being characterized by comprising P: 0-4 wt. %, F: 10-18 wt. %, O: 25-35 wt. %, Si: 10-16 wt. %, Al: 11-19 wt. %, Sr: 20-40 wt. %, La: 0-4 wt. %,
the combined amounts of Al, Sr and F being >48 wt. %, wt. % with respect to the weight of the acid-reactive glass, and the ratio of Al and Si in the acid-reactive glass being greater than 1/1 with respect to weight.

2. The kit of parts in particular according to the preceding claim, the kit of parts comprising Part P and Part L,
Part P being a powder comprising acid-reactive glass,
Part L being a liquid comprising water and polycarboxylic acid,
the polycarboxylic acid comprising a copolymer of acrylic acid and maleic acid,
the acid-reactive glass being characterized by comprising
P: 0-4 wt. %, or 0-3 wt. %,
F: 10-18 wt. %, or 11-16 wt. %,
O: 25-35 wt. %, or 28-34 wt. %,
Si: 10-16 wt. %, or 11-14 wt. %,
Al: 11-19 wt. %, or 12-18 wt. %,
Sr: 20-40 wt. %, or 20-35 wt. %,
La: 0-4 wt. %, or 0-3 wt. %,
the combined amount of Al, Sr and F being >48 wt. %, wt. % with respect to the weight of the acid-reactive glass, and the ratio of Al and Si in the acid-reactive glass being greater than 1/1 with respect to weight.

3. The kit of parts according to claim 1, the acid-reactive glass having a particle size distribution (d90) of 10-15 μm and 1-2 μm (d50).

4. The kit of parts according to claim 1, the acid-reactive glass having been deactivated by treating it with acid, followed by washing with water and drying.

5. The kit of parts according to claim 1, the acid-reactive glass not comprising the following elements alone or in combination: Li, K, Rb, Cs, Be, Mg, each in an amount of more than 0.2 wt. % or 0.1 wt. % with respect to the weight of the acid-reactive glass.

6. The kit of parts according to claim 1, the polycarboxylic acid having a molecular weight Mw in the range of 5,000 to 250,000 g/mol or 10,000 to 100,000 g/mol.

7. The kit of parts according to claim 1, the acrylic acid and maleic acid being present in the polycarboxylic acid in a molar ratio of 35-65% maleic acid to 65-35% acrylic acid, or 40-60% maleic acid to 60-40% acrylic acid.

8. The kit of parts according to claim 1, the kit comprising in addition a complexing agent, the complexing agent being present in the Part L or Part P or Part L and Part P, the complexing agent being preferably selected from tartaric acid, hydroxy butanedionic acid, aldaric acid, phosphono succinic acid and mixtures thereof.

9. The kit of parts according to claim 1, Part P and Part L being provided in a mixing ratio in the range of 6:1 to 1:1 with respect to weight.

10. The kit of parts according to claim 1 comprising in addition a non acid-reactive filler, the non acid-reactive filler being preferably selected from quartz, nitrides, glasses derived from Zr, Sr, C, Sb, Sn, Ba, Zn and Al, borosilicate glasses, kaolin, particles of silica, alumina, titania or zirconia and mixtures thereof, the non acid-reactive filler being present in Part P.

11. The kit of parts according to claim 1, the components being present in the following amounts:
acid-reactive glass: 25 to 86 wt. %,
non acid-reactive filler: 0 to 35 wt. %,
polycarboxylic acid: 3 to 35 wt. %,
water: 2 to 35 wt. %,
complexing agent: 0 to 15 wt. %,
additives: 0 to 10 wt. %,
wt. % with respect to the weight of the composition obtained when combining Part P and Part L.

12. The kit of parts according to claim 1 wherein the components are characterized as follows:
the acid-reactive glass having a particle size distribution (d90) of 10 μm and 2 μm (d50),
the acid-reactive glass having been deactivated by treating it with acid, followed by washing with water and drying,
the polycarboxylic acid having a molecular weight Mw in the range of 10,000 to 100,000 g/mol,
the acrylic acid and maleic acid being present in the polycarboxylic acid in a molar ratio of 35-65% maleic acid and 65-35% acrylic acid or 40-60 maleic acid and 60-40 acrylic acid,
Part P comprising in addition a non acid-reactive glass selected from aluminosilicates.

13. A glass ionomer composition obtainable or obtained by mixing Part P and Part L of the kit of parts of claim 1, the glass ionomer composition being characterized by the following properties alone or in combination after hardening:
flexural strength: 20 MPa to 80 MPa determined according to EN-ISO 9917-2:2010;
compressive strength: 300 to 400 MPa determined according to EN-ISO 9917-1/2007, wherein for covering the composition a glass slab is used instead of a foil;
surface hardness: 150 to 250 MPa determined according to ISO 2039-1.

14. (canceled)

15. (canceled)
